(19) United States
(12) Patent Application Publication
(10) Pub. No.: US 2024/0238568 A1
(43) Pub. Date: Jul. 18, 2024

(54) Balloon Catheter

(71) Applicant: Aldo Tullio Iacono, Manhasset, NY (US)

(72) Inventor: Aldo Tullio Iacono, Manhasset, NY (US)

(21) Appl. No.: 18/219,501

(22) Filed: Jul. 7, 2023

Related U.S. Application Data
(60) Provisional application No. 63/402,262, filed on Jan. 1, 0001.
(60) Provisional application No. 63/479,991, filed on Jan. 14, 2023.

(51) Int. Cl. A61M 25/10; A61B 10/06

(52) U.S. Cl. CPC A61M 25/10185; A61B 10/06; A61M 2025/105; A61M 2025/109

(57) ABSTRACT

A catheter having a plurality of systems, including a first system having a set of pincers for grasping samples, a second system having a port for injecting a fluid that promotes coagulation, a third system wrapped about the first and the second, the proximal end of the third having a port to receive a gas control valve and gas supply; the distal end of the third being made from a material that is flexible where the third provides bidirectional passage of a gas enabling inflation and deflation of the distal end of the third.

### PRIORITY

This application claims priority from Provisional Application No. 63/402,262, entitled Catheter to Biopsy Lung & Tamponade Bleeding After Lung Biopsy filed on 30 Aug. 2022, and Provisional Application No. 63/479,991 entitled Catheter for Pulmonary Hemorrhage filed on 14 Jan. 2023, wherein and incorporates the contents herein by reference.

### BACKGROUND OF THE INVENTION

This invention relates to a catheter, in particular, a catheter having a balloon feature, a biopsy feature, and an injection port feature to improve efficiency and procedural safety while minimizing risk of pulmonary hemorrhage and complications stemming therefrom, including risk of death.

Historically, in order to biopsy the lung, a bronchoscope is deployed into the lung. It is understood, that such biopsy will incur in a certain amount of bleeding therein. However, if the bleeding reaches a certain increased level, the patient may experience respiratory failure from disseminated hemorrhage. Current techniques require the catheter to be removed, and then a second instrument to be inserted into the location of the bleeding, which is extremely challenging, if not impossible. This second instrument having a balloon placing pressure on the location of the bleeding. Moreover, there is the factor of time, and depending on the extent of the bleeding, time is so very precious. On occasion, the bleeding is then minimized if not stopped by the balloon, however, once the balloon is removed, oftentimes the bleeding begins anew.

A transbronchial biopsy is typically performed placing a catheter with a pincer device through the bronchoscope using radiographic methods to navigate to a desired nodule. However, due to the relatively blind nature of the procedure, blood vessels are biopsied along with the desired/targeted tissue thereby increasing the bleeding risk, and risk of fatality.

In this regard, there is a need for a device that is more precise and efficient in limiting the amount of bleeding and complications therefrom, and ultimately, reducing the risk of fatality.

### SUMMARY OF THE INVENTION

The present invention is directed to a catheter having a plurality of features, including a balloon feature having the primary advantage of efficiently and effectively minimizing the amount of blood loss during a biopsy, hence the minimizing the complications therefrom, and ultimately, minimizing the risk of fatality stemming therefrom.

An objective of the present invention includes providing a singular device providing means to biopsy a tissue sample, and concurrently providing means to tamponade bleeding precipitated by the biopsy.

Another objective of the present invention includes providing a singular device that concurrently provides means to inject anticoagulant at the biopsy site, while providing means to tamponade bleeding.

Other objectives, advantages, and novel features of the present invention will become more apparent from the following detailed description when taken in conjunction with the accompanying drawings.

### BRIEF DESCRIPTION OF THE DRAWINGS

The following drawings, in which like reference characters indicate like parts, are provided for illustration of the invention and are not intended to limit the invention in any manner whatsoever.

FIG. 1 illustrates a front view of the preferred embodiment;

FIG. 2 illustrates a magnified view of the junction of the tubes of FIG. 1;

FIG. 3 illustrates a magnified view of the distal end of FIG. 1; and

FIG. 4 illustrates a sectional view of FIG. 1.

### DETAILED DESCRIPTION OF THE INVENTION

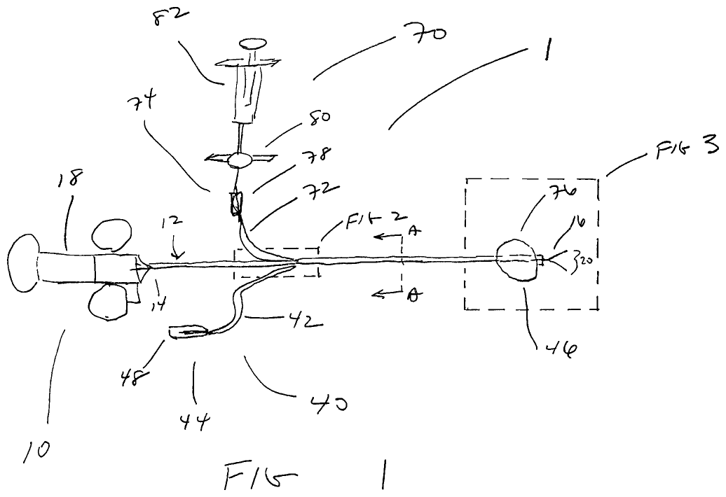

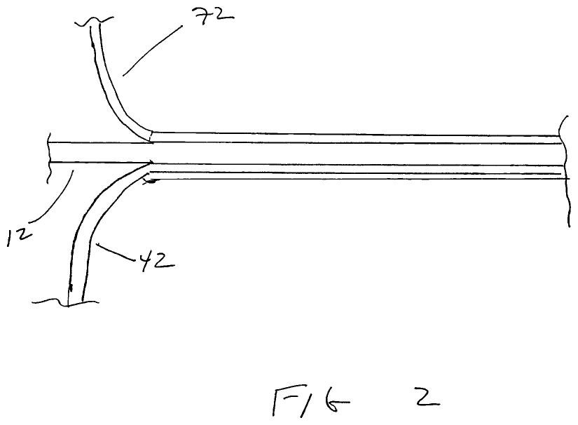

The following descriptions of the preferred embodiments are presented to illustrate the present invention and are not to be construed to limit the claims in any manner whatsoever. In reference to the drawings, namely FIGS. 1 to 4, the preferred embodiment of the present invention is disclosed, which is directed to a catheter 1 having a plurality of systems.

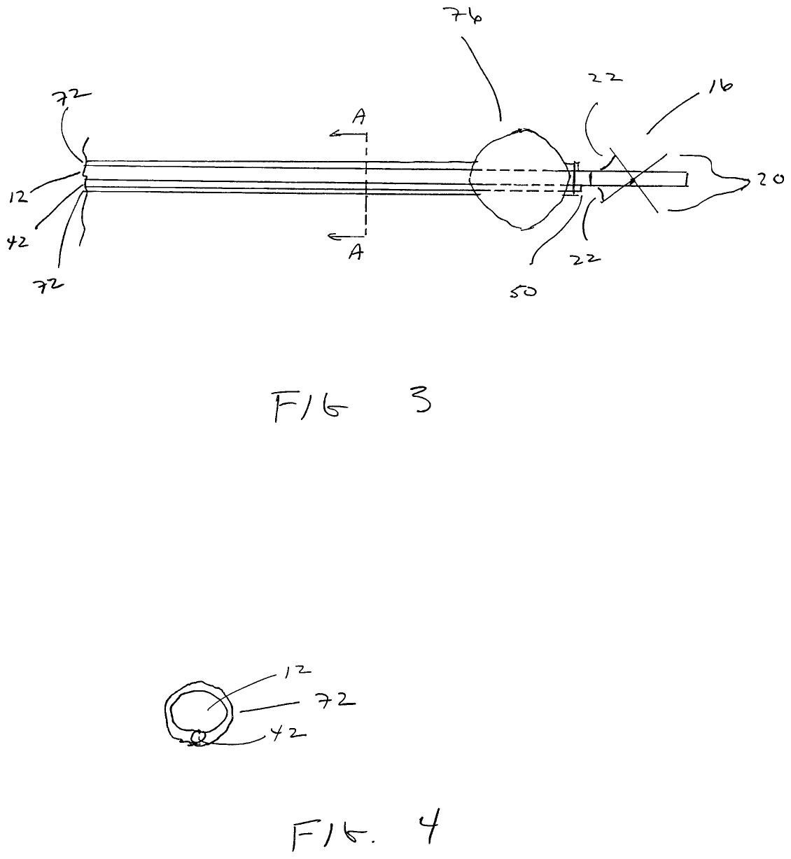

A first system 10 having a tube 12 having a proximal and distal end 14,16, wherein a handle 18 is cooperatively connected to the proximal end 14 of said first tube 12. The distal end 16 having a harvesting means 20 for grasping samples, such as a set of pincers 20, or via cryobiospy, or via a needle (the latter two not shown). The pincers 20 being cooperatively connected to the handle 18 via wires 22 inside said first tube 12. The handle 18 has several positions, including a first position wherein the pincers are closed, a second position wherein the pincers are open, and a third position wherein the pincers are partially open. Pincers 20 are initially closed during the procedure until catheter 1 is in position to grasp a sample from the lung biopsy, wherein the pincers 20 will be opened as illustrated in FIG. 1 and FIG. 3. Once the pincers 20 grasp the targeted biopsy the catheter is pulled manually toward the operator (e.g., the proximal end 14) to disjoin the targeted tissue (specimen) for pathological interpretation. Once the specimen is disjoined, the catheter is left in the airway, the bleeding typically begins, wherein bleeding volumes may exceed 50 CCs, which is massive.

A second system 40 having tube 42 having a proximal and distal end 44, 46, wherein said proximal end 42 includes a port 48 for injecting a fluid that promotes coagulation. The distal end 46 of said second tube 42 includes an opening 50 permitting a bidirectional egress of a fluid. A coagulation fluid can be injected into system 40 via port 48 while the catheter 1 is still in position.

A third system 70 having tube 72 having a proximal and distal end 74,76 wherein said third tube 72 is wrapped about said first 12 and said second tube 42. The proximal end 74 of said third tube 72 having a port 78 to receive a control valve 80 for a gas supply 82. The distal end 76 of said third tube 72 being made from a material that is flexible where said third tube 72 provides bidirectional passage of a gas enabling inflation and deflation of said distal end 76 of said third tube 72 via gas supply 82 via control valve 80.

To further assist in minimizing the bleeding, system 70 can be deployed by inflating the distal end 76 by injecting a gas, e.g., air, oxygen, etc., via the gas supply 82 while the control valve 80 is open. The distal end is sufficiently inflated to contact and put sufficient pressure on the targeted area to stop and/or localize the bleeding and occlusion of the airway.

In practice the catheter 1 is a Cook Captura Disposable Bronchoscope Biopsy Forceps like device 10 that has been modified to include the two additional systems, namely the fluid system 40 and the gas system 70.

The present invention 1 would be placed bronchoscopically and would be inflated into the bleeding pulmonary airway to tamponade bleeding and thus localizing blood within the single bronchial segment and hence avoid respiratory failure from disseminated hemorrhage. A procoagulant injection through the catheter 1 via system 40 after balloon 76 inflation via gas system 70 in the bleeding segment of the lung would localize collagen and other procoagulants to the distal lung beyond the reach of a large bronchoscope. Inflation of the balloon 76 would both tamponade the bleeding, while permitting deep penetration into the distal airway where the bleeding site and injury have occurred (e.g., post alveolar biopsy) without medication regurgitation in the proximal non-bleeding airway, delivered to the arterial bleeding site anatomically and optimally.

More specifically, the inflation via system 70 of the balloon 76 enables medicine such as epinephrine and other procoagulants to be injected via system 40 past the balloon 76 into the bleeding long parenchyma. The catheter 1 is about 2 to 3 millimeters in diameter and about 30 centimeters in length as compared to the much larger bronchoscope.

Moreover, the catheter's novel balloon 76 when inflated via system 70 protects the proximal pulmonary airways from being flooded with blood thereby providing for life saving and sustaining fresh air to be exchanged so that tissue oxygenation and lung function may persist using the catheter 1. In great contrast, in the scenario where the the present invention 1 where not utilized, if blood were to fill all pulmonary alveoli death could occur due to inadequate oxygenation and carbon dioxide removal from the lungs when filled with blood from the uncontrolled bleeding. It should be noted that blood in the lungs typically takes two to three minutes to coagulate during interval the balloon 76 will be inflated via system 70 to stop bleeding, and then deflated via control valve 80 and supply 82 once bleeding ceases. Furthermore, bleeding occurs in about three to five percent of all biopsy procedures in the lung by transbronchial biopsy, a common procedure used to diagnose lung cancer and other lung illnesses. Lastly, spontaneous bleeding may occur in the lungs from common causes such as cancer, infection, inflammatory diseases, and general anti-coagulation commonly used to prevent blood clots.

It is envisioned that the above three systems, namely, system 10, system 40, and system 70 can be condensed into a two-system catheter by combining the first system 10, with the second system 40 (although combined systems are not shown).

All of the above referenced patents; patent applications and publications are hereby incorporated by reference. Many variations of the present invention will suggest themselves to those of ordinary skill in the art in light of the above detailed description. All such obvious modifications are within the full-intended spirit and scope of the claims of the present application.

What is claimed is:

1. A catheter comprising:
a plurality of systems, wherein a first system includes a first tube having a proximal and distal end, wherein a handle is cooperatively connected to the proximal end of said first tube, said distal end having a set of pincers for grasping samples, wherein said pincers being cooperatively connected to said handle via wires inside said first tube, wherein said handle has several positions, including a first position wherein the pincers are closed, a second position wherein the pincers are open, and a third position wherein the pincers are partially open;
a second system includes a second tube having a proximal and distal end, wherein said proximal end includes a port for injecting a fluid that promotes coagulation, wherein said distal end of said second tube includes an opening permitting a bidirectional egress of a fluid;
a third system includes a third tube having a proximal and distal end wherein said third tube is wrapped about said first and said second tube, said proximal end of said third tube having a port to receive a gas supply and gas control valve; and
said distal end of said third tube being made from a material that is flexible where said third tube provides bidirectional passage of a gas enabling inflation and deflation of said distal end of said third tube.

2. A catheter comprising:
a plurality of systems, a first system providing means for procuring a biopsy from a desired location, a second system providing means for administering medicine to the desired location, and a third system providing means to tamponade the targeted location.

3. A catheter as in claim 2, wherein said first system comprises a first tube having a proximal and distal end, wherein a handle is cooperatively connected to the proximal end of said first tube, said distal end having a harvesting means for grasping samples selected from the group consisting of a) a set of pincers; b) cryobiopsy; and c) a needle.

4. A catheter as in claim 3, wherein said pincers being cooperatively connected to said handle via wires inside said first tube.

5. A catheter as in claim 4, wherein said handle has several positions, including a first position wherein the pincers are closed, a second position wherein the pincers are open, and a third position wherein the pincers are partially open.

6. A catheter as in claim 2, wherein said second system comprises a second tube having a proximal and distal end, wherein said proximal end includes a port for injecting a fluid that promotes coagulation.

7. A catheter as in claim 6, wherein said distal end of said second tube includes an opening permitting a bidirectional egress of a fluid.

8. A catheter as in claim 2, wherein said third system comprises a third tube having a proximal and distal end wherein said third tube is wrapped about said first and said second tube.

9. A catheter as in claim 8, said proximal end of said third tube having a port to receive a gas control valve and a gas supply.

10. A catheter as in claim 8, wherein said distal end of said third tube being made from a material that is flexible.

11. A catheter as in claim 8, where said third tube provides bidirectional passage of a gas enabling inflation and deflation of said distal end of said third tube.
